(19) United States
(12) Patent Application Publication
(10) Pub. No.: US 2026/0014009 A1
(43) Pub. Date: Jan. 15, 2026

(54) System and Method for Managing Weight Loss Using an Intraoral Device and Software

(71) Applicant: Jordan Pierce Hidalgo, Belleair, FL (US)

(72) Inventor: Jordan Pierce Hidalgo, Belleair, FL (US)

(21) Appl. No.: 18/769,791

(22) Filed: Jul. 11, 2024

(51) Int. Cl. A61F 5/00; A61B 5/00; G09B 5/06; G16H 20/60

(52) U.S. Cl. CPC A61F 5/0006; A61B 5/4833; A61B 5/682; A61B 5/7267; G09B 5/065; G16H 20/60

(57) ABSTRACT

An integrated weight management system comprising a biocompatible intraoral device and personalized software is disclosed. The device, configured for insertion into the user's mouth, creates a separation between the tongue and teeth causing the stomatognathic system, limiting hunger cues associated with food anticipation. Sensors coupled to the device detect its removal and insertion, enabling the system to track adherence to meal plans and weight loss progress. The software utilizes machine learning algorithms to generate personalized meal plans, food intake timing, nutrition education, and exercise routines based on user preferences, dietary restrictions, and weight loss goals. The system analyzes user data to provide progress reports, adjust meal plans, and send alerts to promote accountability and compliance. This comprehensive approach addresses both physiological and psychological aspects of weight management, offering a novel and effective solution for achieving and maintaining a healthy weight.

### BACKGROUND

Obesity and overweight conditions have become a significant health concern worldwide, leading to an increased risk of various chronic diseases such as diabetes, cardiovascular disorders, and certain types of cancer. Conventional weight loss methods, including dieting and exercise, often fail due to a lack of discipline and adherence to the prescribed regimens. While some weight loss programs and apps aim to address this issue by providing meal plans and tracking tools, they do not effectively tackle the fundamental problem of limiting food intake.

In the field of weight loss devices, various oral appliances have been developed to help individuals control their food consumption. For example, U.S. Patent Application Publication No. US20160067073A1 discloses a weight loss device comprising at least one peripheral plate fixed to the dental arch and supporting a central plate that rests against the palate or floor of the mouth to limit tongue movements [1]. However, this device focuses primarily on restricting tongue motion and does not provide a comprehensive solution that addresses the sensory cues associated with eating and the need for personalized nutrition education and support.

The present invention aims to overcome the limitations of existing weight loss methods and devices by providing a novel system that combines an intraoral device with intelligent software to effectively manage weight loss. The proposed system not only creates a physical barrier to food intake but also leverages machine learning algorithms to create personalized meal plans, provide nutrition education, and track adherence to the program.

### SUMMARY

The present invention addresses the limitations of existing weight loss methods by providing a comprehensive system that combines a physical device for limiting food intake with personalized software for creating meal plans, tracking progress, and delivering educational content.

The system comprises a biocompatible physical device configured to be inserted into the user's mouth, creating a separation between the tongue and teeth. This separation interrupts the sensory check by the stomatognathic system, thereby limiting hunger cues associated with the anticipation of food. The device can be custom-fitted to the user's mouth and is removable for ease of use.

Paired with the physical device is a software program that utilizes machine learning algorithms to generate personalized meal plans and food intake timing based on the user's preferences, dietary restrictions, and weight loss goals. The software also provides nutrition education videos and standardized exercise routines adapted to the user's fitness level and physical limitations.

Sensors coupled to the physical device detect its removal and insertion, enabling the system to track the user's adherence to the meal plans and weight loss progress. The software program analyzes this data to provide progress reports and adjust the meal plans as needed. Alerts are sent to the user's mobile device when the physical device is removed for an extended period, promoting accountability and compliance.

The present invention offers several advantages over existing weight loss solutions. By integrating a physical barrier to food intake with personalized software, the system addresses both the physiological and psychological aspects of weight management. The machine learning algorithms ensure that the meal plans and educational content are tailored to the individual user, increasing the likelihood of successful weight loss.

Furthermore, the invention's mobile application allows users to access their meal plans, educational resources, and progress reports conveniently. The application also enables users to input their adherence to the program, providing valuable data for the machine learning algorithms to refine the system's effectiveness continually.

In summary, the present invention provides a novel and comprehensive approach to weight management by combining a physical device for limiting food intake with personalized software for creating meal plans, tracking progress, and delivering educational content. This integrated system addresses the limitations of existing weight loss methods and offers a more effective solution for achieving and maintaining a healthy weight.

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

### BRIEF DESCRIPTION OF THE DRAWINGS

The various exemplary embodiments of the present invention. which will become more apparent as the description proceeds, are described in the following detailed description in conjunction with the accompanying drawings, in which:

FIG. 1 illustrates the key components and data flow of the weight management system.

FIG. 2 provides a cross-sectional view of the physical intraoral device when inserted into the user's mouth.

FIG. 3 illustrates a simple embodiment of the progress tracking interface of the weight.

FIG. 4 is a flow diagram illustrating a method for managing weight loss using the physical intraoral device.

### DETAILED DESCRIPTION

In the following detailed description of the preferred embodiments, reference is made to the accompanying drawings, which form a part hereof and show, by way of illustration, specific embodiments in which the invention may be practiced. It is to be understood that other embodiments may be used and structural or logical changes may be made without departing from the scope of the present invention. The following detailed description, therefore, is not to be taken in a limiting sense, and the scope of the present invention is defined by the appended claims.

The following description is provided as an enabling teaching of the present systems, and/or methods in its best, currently known aspect. To this end, those skilled in the relevant art will recognize and appreciate that many changes can be made to the various aspects of the present systems described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

The terms “a” and “an” and “the” and similar references used in the context of describing a particular embodiment of the present invention (especially in the context of certain claims) are construed to cover both the singular and the plural. The recitation of ranges of values herein is merely intended to serve as a shorthand method of referring individually to each separate value falling within the range. Unless otherwise indicated herein. each individual value is incorporated into the specification as if it were individually recited herein.

All systems described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word or as used herein means any one member of a particular list and also includes any combination of members of that list. Further, one should note that conditional language, such as, among others, “can,” “could,” “might.” or “may.” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

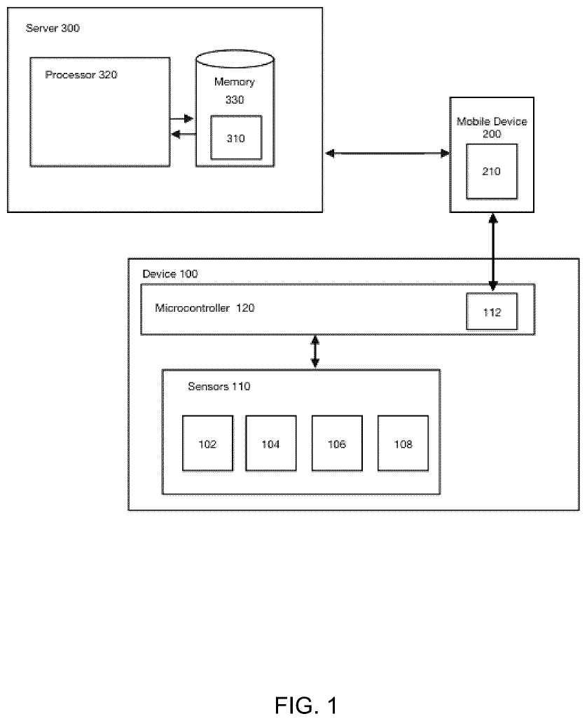

FIG. 1 illustrates the key components and data flow of the weight management system. The system includes a physical intraoral device 100 configured to be inserted into the user's mouth to limit food intake. The physical device 100 incorporates an array of sensors 110, such as accelerometers 102, pressure sensors 104, proximity sensors 106 and temperature sensors 108. These sensors 110 are coupled to a microcontroller unit (MCU) 120 that collects and processes the sensor data.

The MCU 120 includes a wireless transceiver, such as a Bluetooth Low Energy (BLE) module 112, to transmit the sensor data to a paired mobile device 200 running a dedicated mobile application 210. The mobile application 210 communicates with a remote server 300 that executes a weight management software program 310.

The software program 310 utilizes machine learning algorithms to analyze the sensor data along with user-inputted information to generate personalized meal plans, food intake schedules, nutrition education videos, and standardized exercise routines. The server 300 includes a processor 320 to execute the software program 310 and a memory 330 to store the program code, machine learning models, and user data.

The mobile application 210 displays the personalized guidance and content to the user and allows them to input their adherence and progress data. This data is sent back to the server 300 for the software program 310 to track the user's progress and iteratively refine the guidance. The software program 310 can detect if the user has removed the physical device 100 for an extended period based on an absence of sensor data, and prompt the mobile application 210 to display an alert.

In some embodiments the machine learning system architecture is employed by the weight management software program (310). The system leverages a feedback loop that continuously improves the accuracy and effectiveness of the personalized meal plans, exercise routines, and weight loss predictions. The machine learning process begins with the collection of user data, including demographic information, dietary preferences, health conditions, and physical limitations. This data is securely stored in a PostgreSQL database on the remote server 300 and preprocessed using Python libraries such as Pandas and NumPy to ensure data quality and consistency.

The preprocessed data is then fed into various machine learning models, such as decision trees, support vector machines (SVM), and deep neural networks (DNN), which are trained to generate personalized meal plans, exercise routines, and weight loss predictions. The models are implemented using popular machine learning frameworks such as TensorFlow and PyTorch, and are continuously refined using techniques such as cross-validation and hyperparameter tuning.

As users interact with the mobile application 210 and provide feedback on the generated meal plans and exercise routines, the machine learning system incorporates this new data into its training process. This feedback loop allows the models to learn from user preferences and adapt their recommendations accordingly, improving the overall effectiveness of the weight management program. The machine learning system also employs natural language processing (NLP) techniques to analyze user-generated text data, such as food preferences and dietary restrictions. NLP libraries such as spaCy and NLTK are used to perform tasks such as tokenization, part-of-speech tagging, and named entity recognition, enabling the system to extract meaningful insights from unstructured text data.

The output of the machine learning models is then transmitted back to the mobile application (210) via a RESTful API, where it is presented to the user in the form of personalized meal plans, exercise routines, and weight loss predictions. The API is secured using industry-standard authentication and authorization protocols, such as OAuth 2.0 and JSON Web Tokens (JWT), to ensure the privacy and integrity of user data.

In certain embodiments, the intraoral device 100 and associated weight management system address different types of hunger to facilitate weight loss. By physically limiting food intake, the device 100 helps manage sensory hunger triggered by the sight, smell, or thought of food. The personalized guidance from the software program 310, displayed on the mobile application 210, aids in controlling emotional hunger caused by stress, boredom, or other psychological factors. The system therefore targets sensory and emotional hunger, rather than physical hunger characterized by stomach growling, to promote sustainable weight management.

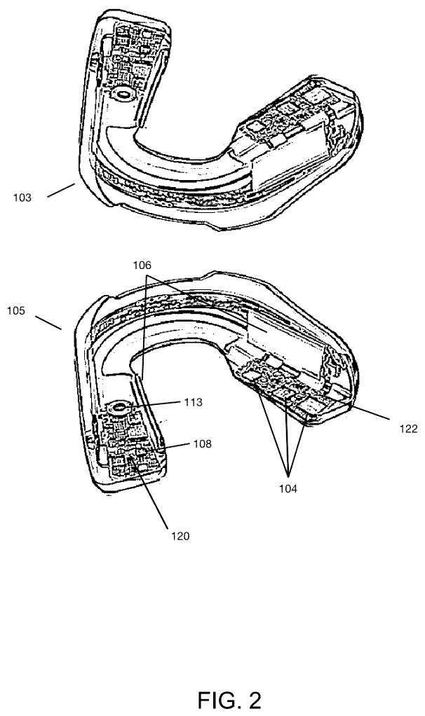

FIG. 2 provides a perspective view of the physical intraoral device 100 when. The device is composed of a biocompatible elastomeric material, such as medical-grade silicone, that can be compression molded into a custom-fit appliance based on a dental impression or intraoral scan of the user's teeth and gums.

The device 100 has an upper portion 103 that fits over the maxillary teeth and a lower portion 105 that fits over the mandibular teeth. The upper portion 103 and lower portion 105 are connected by an adjustable connector such as an elastic (not shown) that allows the vertical separation between the portions to be increased or decreased. This enables the device 100 to accommodate different levels of food restriction.

When inserted, the device 100 occupies the interocclusal space between the upper and lower teeth, creating a physical barrier that prevents the tongue from contacting the teeth and palate during mastication and swallowing. This physical separation interrupts the sensory stimuli and anticipatory cues from the lingual nerve and subsequently the stomatognathic system, thereby reducing hunger sensations and cravings.

The cross-section also shows the various sensors 110 within the physical intraoral device 100. The device 100 incorporates an inertial measurement unit (IMU) 113 containing a 3-axis accelerometer and 3-axis gyroscope. The IMU 112 detects the orientation and motion of the device 100, such as during insertion, removal, or mastication.

Pressure sensors 104, such as force-sensitive resistors or capacitive sensors, are embedded at key points on the interocclusal surface of the device 100. These sensors 104 measure the magnitude and distribution of bite forces exerted by the user.

Proximity sensors 106, such as infrared (IR) transceivers, are located on the buccal and lingual flanges of the device 100. These sensors 106 detect the proximity of the device 100 to the adjacent oral mucosa and can infer if the device is fully seated in the correct position.

Temperature sensors 108, such as negative temperature coefficient (NTC) thermistors, are also distributed throughout the device 100. These sensors 108 measure the intraoral temperature to detect if the device is being worn by the user. The intraoral temperature is typically a few degrees above ambient temperature.

The sensors 110 are connected to the MCU 120 via a printed circuit board (PCB) 122 that is overmolded into the elastomeric material. The PCB 122 provides power regulation and signal conditioning for the sensors. The MCU 120 includes an analog-to-digital converter (ADC) to sample the sensor signals and a BLE transceiver (not shown) to wirelessly transmit the sensor data to the paired mobile device 200.

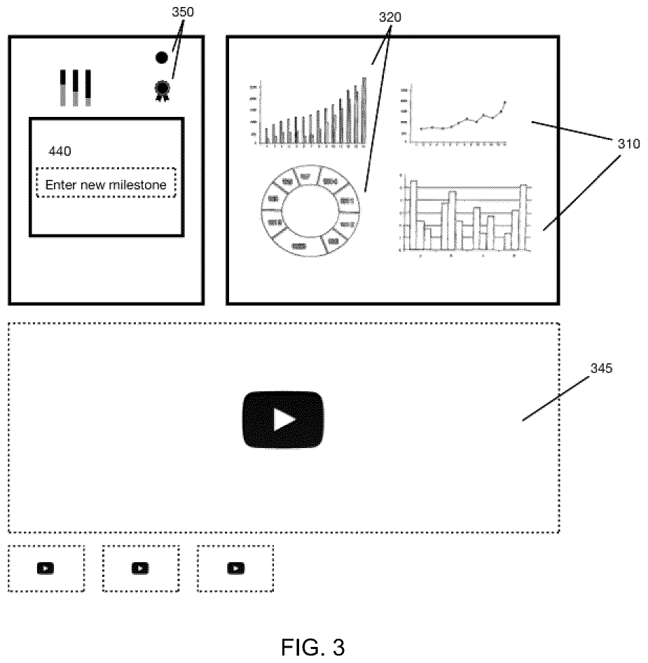

FIG. 3 illustrates a simple embodiment of the progress tracking interface of the weight management software program (310) accessible through the mobile application (210) installed on the user's mobile device (200). The software program (310) also provides a comprehensive and personalized weight loss solution, incorporating features such as meal planning, nutrition education, exercise routines, progress tracking, and community support.

The progress tracking interface is a key component of the weight management software program (310), providing users with a visual representation of their weight loss journey. The interface includes interactive graphs 310 and charts 320 that display the user's weight loss progress, BMI changes, and adherence to meal plans over time. Users can set weight loss milestones 340 and receive virtual badges and rewards 350 for achieving them, leveraging gamification techniques to maintain motivation and engagement.

In some embodiments the weight management software program (310) may include additional user interfaces and functionalities comprising:
An onboarding process where the user inputs their dietary preferences, allergies, and any physical limitations. This information is captured using intuitive form-based user interfaces (UI) with checkboxes, dropdown menus, and text input fields The mobile application (210) securely transmits this data to the remote server (300) via HTTPS protocol, where it is processed by the weight management software program (310) to generate personalized meal plans and exercise routines.

A meal planning interface (not shown) which allows users to view their daily, weekly, and monthly meal plans, which are generated based on their preferences and nutritional requirements. A meal plans presented in, a card-based layout, with high-resolution images of each meal and detailed nutritional information. Users tap on a meal card to view the recipe, ingredients, and step-by-step cooking instructions. The software program (310) utilizes natural language processing (NLP) techniques, such as named entity recognition and sentiment analysis, to analyze user preferences and generate meal plans that align with tastes and dietary needs.

A nutrition education section (not shown) features a library of personalized video content, tailored to the user's specific health conditions and dietary preferences. The videos are categorized by topic and difficulty level, allowing users to easily navigate and find content relevant to their needs. The software program (310) employs machine learning algorithms, such as collaborative filtering and content-based recommendation systems, to suggest videos based on the user's viewing history and preferences.

An exercise video interface 345 provides users with a collection of standardized exercise routines adapted to their fitness level and physical limitations. The routines are demonstrated through high-quality video content, with options to filter by workout type, duration, and difficulty. Users can track their progress within the application, logging completed workouts and viewing their activity history in a calendar view.

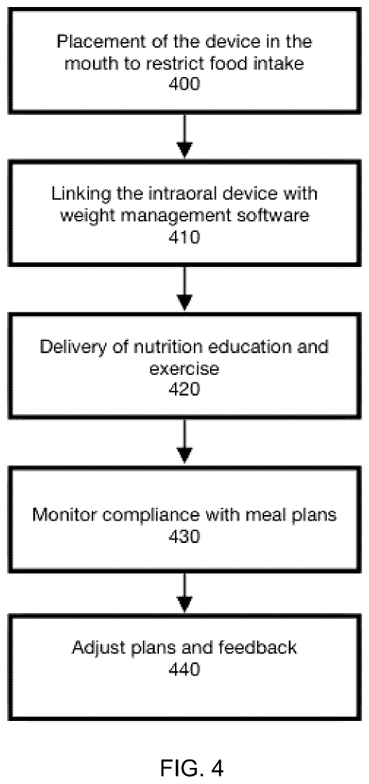

FIG. 4 is a flow diagram illustrating a method for managing weight loss using the physical intraoral device 100 in conjunction with the weight management software program 310.

The method begins at step 400 with inserting the physical intraoral device 100 into a user's mouth. The physical intraoral device 100 is configured to prevent intake of food by creating a physical separation between the user's tongue and teeth, thereby limiting hunger cues by interrupting sensory experiences associated with anticipation of food.

At step 410, the physical intraoral device 100 is paired with the weight management software program 310 comprising machine learning algorithms executed by the processor 320. The machine learning algorithms analyze the user's profile data stored in the memory 330 to create personalized meal plans and optimize food intake timing for the user.

The method proceeds to step 420, where the weight management software program 310 provides the user with nutrition education videos and standardized exercise routines via the mobile application 210 on the mobile device 200. The videos and exercise routines are designed to assist the user in improving their overall quality of life while managing their weight loss journey.

At step 430, the user's adherence to the personalized meal plans and their weight loss progress is tracked using the sensors 110 integrated into the physical intraoral device 100. The sensors 110 may include inertial measurement units (IMUs) 112, pressure sensors 114, proximity sensors 116, and temperature sensors 118. These sensors are configured to detect the removal and insertion of the physical intraoral device 100, allowing the weight management software program 310 to monitor the user's compliance with the prescribed meal plans and device usage.

The sensor data is transmitted from the physical intraoral device 100 to the mobile device 200 via the BLE transceiver 124 on the printed circuit board (PCB) 122 and the microcontroller unit (MCU) 120. The mobile application 210 then sends the data to the remote server 300 for analysis by the weight management software program 310.

Based on the tracked data, the weight management software program 310 may adjust the personalized meal plans and provide feedback to the user through the mobile application 210, as shown in step 440. This feedback loop allows for continuous optimization of the user's weight loss plan based on their individual progress and adherence to the program.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A system for managing weight loss, the system comprising:
a physical device configured to be inserted into a user's mouth, wherein the physical device prevents intake of food by creating a physical separation between the user's tongue and teeth, thereby interrupting a sensory check by the stomatognathic system and limiting hunger cues;
a memory storing a software program comprising machine learning algorithms;
a processor configured to execute the software program to:
create personalized meal plans and food intake timing for the user, provide nutrition education videos and standardized exercise routines, and track the user's adherence to the meal plans and weight loss progress;
one or more sensors coupled to the physical device, the sensors configured to detect removal and insertion of the physical device;
wherein the processor is further configured to receive data from the one or more sensors to track the user's adherence to the meal plans and weight loss progress.

2. The system of claim 1, wherein the physical device is constructed from a biocompatible material selected from the group consisting of silicone, polyethylene, and polypropylene.

3. The system of claim 1, wherein the physical device is custom-fitted to the user's mouth based on dental impressions.

4. The system of claim 1, wherein the physical device is configured to be removably inserted into the user's mouth.

5. The system of claim 1, wherein the one or more sensors comprise at least one of an accelerometer, a pressure sensor, and a proximity sensor, and a temperature sensor.

6. The system of claim 1, wherein the processor is further configured to transmit alerts to the user's mobile device when the one or more sensors detect that the physical device has been removed for longer than a predetermined time period.

7. The system of claim 1, wherein the processor is further configured to adjust the personalized meal plans based on the user's adherence and weight loss progress.

8. The system of claim 1, wherein the nutrition education videos are customized based on the user's dietary preferences and restrictions.

9. The system of claim 1, wherein the standardized exercise routines are adapted based on the user's fitness level and physical limitations.

10. The system of claim 1, further comprising a mobile application configured to display the personalized meal plans, nutrition education videos, and exercise routines to the user.

11. The system of claim 10, wherein the mobile application is further configured to receive user input regarding adherence to the meal plans and exercise routines.

12. The system of claim 1, wherein the processor is further configured to generate progress reports summarizing the user's adherence to the meal plans and weight loss progress over time.

13. The system of claim 1, wherein the machine learning algorithms are trained on data from a plurality of users to continuously improve the personalized meal plans and weight loss predictions.

14. A method for managing weight loss, the method comprising:
inserting a physical device into a user's mouth, wherein the physical device is configured to prevent intake of food by creating a physical separation between parts of the user's mouth, thereby limiting hunger cues by interrupting sensory experiences associated with anticipation of food;
pairing the physical device with a software program comprising machine learning algorithms to create personalized meal plans and food intake timing for the user;
providing the user, through the software program, nutrition education videos and standardized exercise routines to assist in improving overall quality of life; and
tracking the user's adherence to the meal plans and weight loss progress using sensors configured to detect removal and insertion of the physical device.

15. The method of claim 14, wherein the physical device is constructed from a biocompatible material selected from the group consisting of silicone, polyurethane, and thermoplastic elastomers.

16. The method of claim 14, wherein the physical device is custom-fitted to the user's mouth using 3D scanning and printing technologies.

17. The method of claim 14, wherein the software program is accessible through a mobile application installed on the user's smartphone or tablet device.

18. The method of claim 14, wherein the physical device is configured to be removably inserted into the user's mouth.

19. The method of claim 14, further comprising transmitting alerts to the user's mobile device when the sensors detect that the physical device has been removed for longer than a predetermined time period.

20. The method of claim 14, further comprising adjusting the personalized meal plans based on the user's adherence and weight loss progress using the machine learning algorithms.

21-32. (canceled)
